(19) United States
(12) Patent Application Publication
(10) Pub. No.: US 2009/0062361 A1
(43) Pub. Date: Mar. 5, 2009

(54) THERAPEUTIC HYDANTOINS

(75) Inventor: David W. Old, Irvine, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/192,263

(22) Filed: Aug. 15, 2008

Related U.S. Application Data
(60) Provisional application No. 60/968,941, filed on Aug. 30, 2007.

(51) Int. Cl. A61K 31/427; C07D 409/06; A61P 27/02

(52) U.S. Cl. 514/391; 548/315.1

(57) ABSTRACT

Disclosed herein is a compound represented by a formula

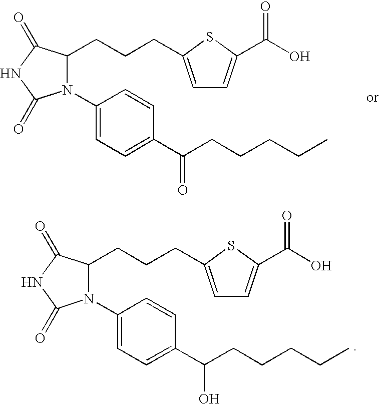

Therapeutic methods, compositions, and medicaments related thereto are also disclosed.

### CROSS-REFERENCE TO RELATED APPLICATIONS

This application claims priority to U.S. Provisional Patent Application No. 60/968,941, filed Aug. 30, 2007, incorporated by reference herein.

### DESCRIPTION OF THE INVENTION

Disclosed herein is a compound having a formula

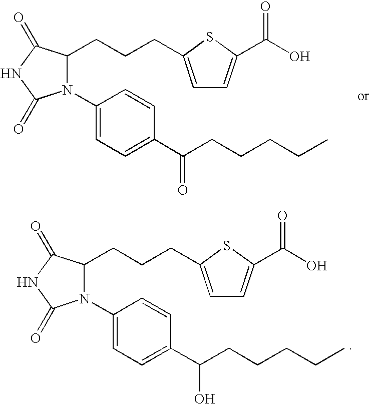

One embodiment is a compound represented by a formula:

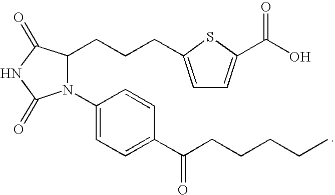

Another embodiment is a compound represented by a formula:

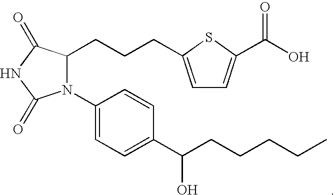

Another embodiment is a compound represented by a formula:

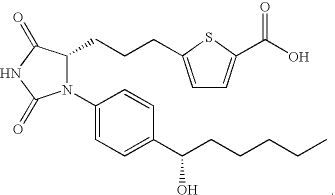

Another embodiment is a compound represented by a formula:

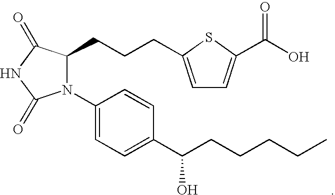

Another embodiment is a compound represented by a formula:

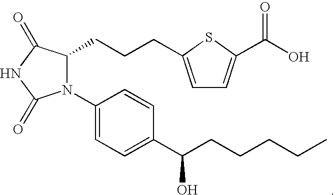

These compounds are useful for the treatment of glaucoma and the reduction of intraocular pressure. The compound is incorporated into a dosage form or a medicament and administered to the mammal, such as a person, in need thereof. For example, a liquid composition may be administered as an eye drop or a solid or liquid dosage form may also be administered orally. Other types of dosage forms and medicaments are well known in the art, and may also be used here.

Another embodiment is a composition comprising a compound disclosed herein, wherein said composition is a liquid which is ophthalmically acceptable.

Another embodiment is a medicament comprising a compound disclosed herein, wherein said medicament is a liquid which is ophthalmically acceptable.

Another embodiment is a method comprising administering a compound disclosed herein to a mammal for the reduction of intraocular pressure.

Another embodiment is use of a compound disclosed herein in the manufacture of a medicament for the treatment of glaucoma or ocular hypertension.

Another embodiment is a kit comprising a composition comprising compound disclosed herein, a container, and instructions for administration of said composition to a mammal for the treatment of glaucoma or elevated intraocular pressure.

Methods of formulating compounds such as those disclosed herein for ophthalmic and other pharmaceutical preparations are well known in the art. For example, U.S. patent application Ser. No. 10/599,046, filed on Sep. 18, 2006, incorporated by reference herein, describes typical formulation methods.

For the purposes of this disclosure, “treat,” “treating,” or “treatment” refer to the use of a compound, composition, therapeutically active agent, or drug in the diagnosis, cure, mitigation, treatment, or prevention of disease or other undesirable condition.

Unless otherwise indicated, reference to a compound should be construed broadly to include pharmaceutically acceptable salts, prodrugs, tautomers, alternate solid forms, non-covalent complexes, and combinations thereof, of a chemical entity of the depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids), one or more protonated basic groups (e.g. amines), or both (e.g. zwitterions).

A prodrug is a compound which is converted to a therapeutically active compound after administration. For example, conversion may occur by hydrolysis of an ester group, such as a C₁₋₆ alkyl ester of the carboxylic acid group of the present compounds, or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

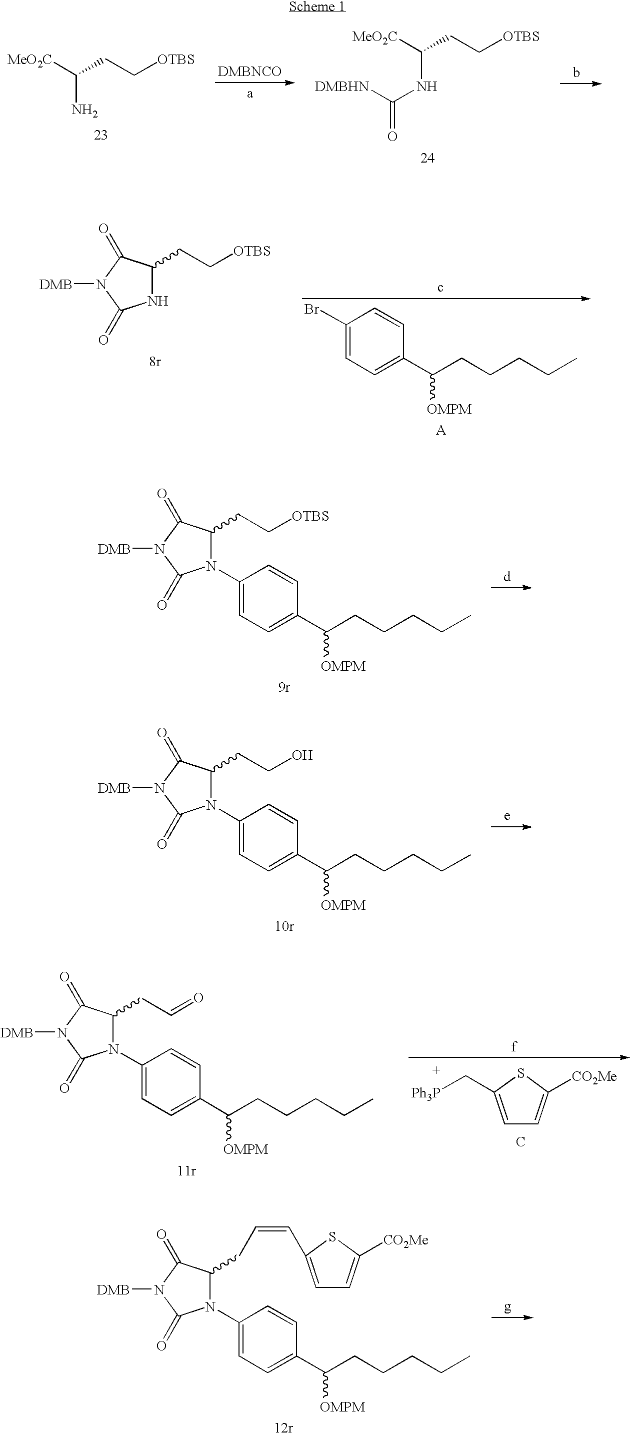

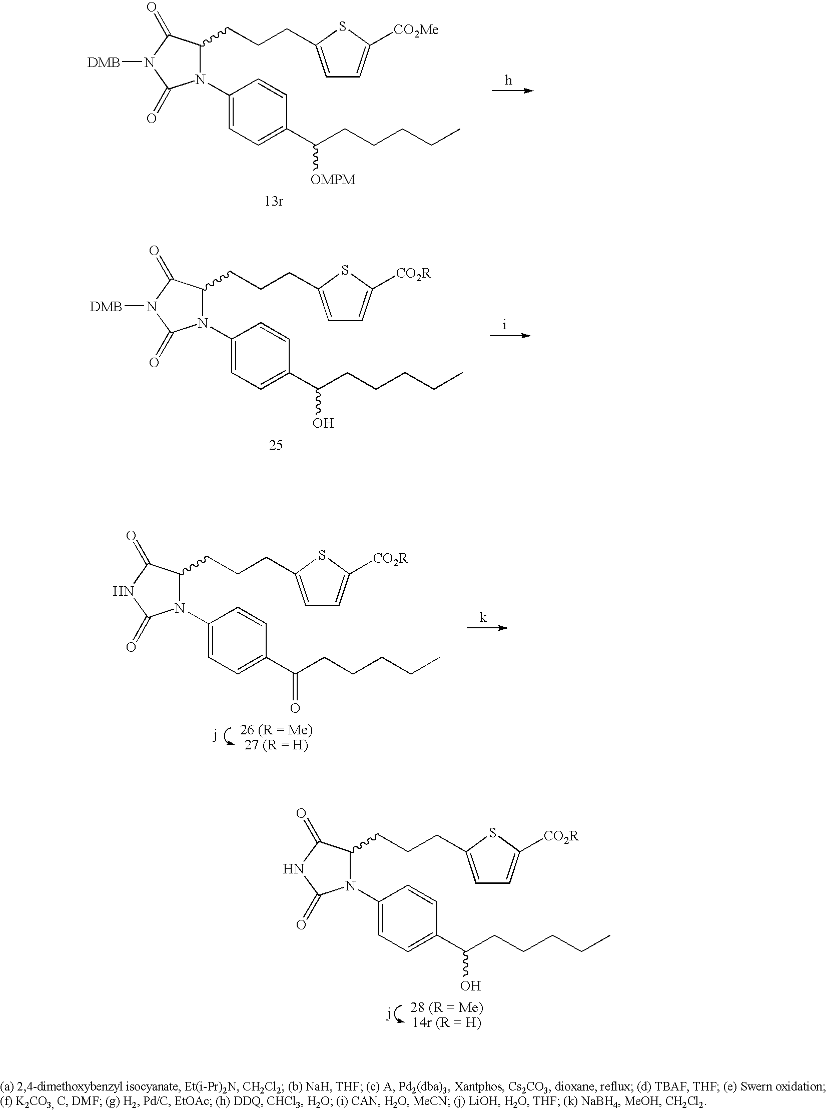

### EXAMPLE 1

### 5-(3-(3-(4-hexanoylphenyl)-2,5-dioxoimidazolidin-4-yl)propyl)thiophene-2-carboxylic acid (27)

Step 1. Addition of 23 to DMB-NCO to give 24

A solution of amino acid 23 (see Kline, T., et al. J. Med. Chem. 2002, 45, 3112-3129; 12.35 g, 49.9 mmol) and diisopropylethylamine (12.9 g, 99.8 mmol) in CH₂Cl₂ (100 mL) was added over 30 min to a solution of 2,4-dimethoxybenzyl isocyanate (see Trost and Fandrick, Org. Lett. 2005, 7, 823-826; 9.65 g, 49.9 mmol) in CH₂Cl₂ (100 mL) at room temperature. After stirring 1 h, the reaction mixture was diluted with CH₂Cl₂ (100 mL) and washed with water (2×150 mL), brine (150 mL), filtered through filter paper and concentrated in vacuo to afford 22 g of an oil. This oil was dissolved in hot hexane (250 mL) and stirred at room temperature overnight. The precipitated solid was collected by filtration. After drying under vacuum, 12.5 g of solid 24 was obtained which was shown by HPLC to be approximately 86% pure. The filtrate was concentrated to afford 9.5 g of an oil. This oil was purified by flash chromatography on 150 g silica gel (CH₂Cl₂→15% EtOAc/CH₂Cl₂, gradient) to afford 1.8 g of 24 as an oil that was shown by HPLC to be approximately 90% pure. The two samples of impure 24 were combined and purified by flash chromatography on 150 g silica gel (CH₂Cl₂→20% EtOAc/CH₂Cl₂, gradient) to afford 12.4 g (56%) of 24 as an oil that solidified on standing.

Step 2. Cyclization of 24 to give 8r

Sodium hydride (75 mg, 3.1 mmol) was added in one portion to a solution of urea 24 (12.3 g, 27.9 mmol) in THF (150 mL) at room temperature. After 15 min the reaction mixture was diluted with EtOAc (150 mL), washed with water (2×100 mL) and brine (100 mL), filtered through filter paper and concentrated to afford 11 g of an oil. This oil was purified by flash chromatography on 125 g silica gel (CH₂Cl₂→10% EtOAc/CH₂Cl₂, gradient) to afford, after drying in vacuo 8.8 g (77%) of 8r as a solid. This material showed an optical rotation of 0° and is presumed to be racemic.

Step 3. Arylation of 8r with A to give 9r

Pd₂(dba)₃ (41 mg, 0.045 mmol), Xantphos (77 mg, 0.133 mmol) and Cs₂CO₃ (428 mg, 1.31 mmol) were added sequentially to a solution of 8r (450 mg, 1.10 mmol) and bromoarene A (see Old and Dinh, WO2006/098918, incorporated by reference herein; 372 mg, 0.99 mmol) in 1,4-dioxane (7.1 mL). The flask was fitted with a reflux condenser, evacuated and refilled with nitrogen (5×) then heated at reflux. After 18 h, the reaction was cooled, diluted with EtOAc (75 mL) and filtered through celite, washing with excess EtOAc. The EtOAc filtrate was concentrated in vacuo. The crude residue was purified on 120 g silica gel (hexanes→25% EtOAc/hexanes, gradient) to afford 630 mg (91%) of 9r.

Step 4. Deprotection of 9r to give 10r

Tetrabutylammonium fluoride (2.7 mL of a 1.0 M solution in THF, 2.7 mmol) was added to a solution of 9r (630 mg, 0.89 mmol) in THF (6 mL) at 0° C. The cooling bath was removed and the reaction was allowed to stir at room temperature overnight. After 18 h at room temperature the reaction was concentrated in vacuo. Water (50 mL) was added and the mixture was extracted with EtOAc (3×80 mL). The combined extracts were washed with brine (50 mL), dried (Na₂SO₄), filtered and concentrated in vacuo. The crude residue was purified on 40 g silica (hexanes→60% EtOAc/hexanes, gradient) to afford 465 mg (88%) of 10r.

Step 5. Oxidation of 10r to give 11r

DMSO (69 μL, 0.97 mmol) was added to a solution of oxalyl chloride (234 μL of a 2.0 M solution in CH₂Cl₂, 0.47 mmol) and CH₂Cl₂ (3.3 mL) at −78° C. After 15 min at −78° C., a solution of 10r (230 mg, 0.39 mmol) in CH₂Cl₂ (1.1 mL) was added via cannula. After 15 at −78° C., triethylamine (434 μL, 3.11 mmol) was added. After 15 min, the reaction was allowed to warm to 0° C. After 30 min at room temperature, the reaction was allowed to warm to room temperature then saturated aqueous NaHCO₃ (30 mL) was added. The mixture was extracted with CH₂Cl₂ (3×50 mL), then dried (Na₂SO₄), filtered and concentrated in vacuo. Crude 11r was taken on to the next step without further purification.

Step 6. Reaction of 11r with C to give 12r

Potassium carbonate (99.99%, 538 mg, 3.90 mmol) was added to crude 11r (˜0.39 mmol) and phosphonate C (see Collect. Czech. Chem. Commun. 1994, 58, 138-148; 200 mg, 0.40 mmol) in DMF (3.9 mL) at room temperature. The reaction mixture was stirred at room temperature for 3 d then diluted with water (30 mL) and extacted with EtOAc (150 mL). The organic phase was washed with water (2×50 mL) and brine (50 mL) then dried (Na₂SO₄), filtered and concentrated in vacuo. The crude residue was purified on 40 g silica (hexanes→50% EtOAc/hexane, gradient) to afford 163 mg (58% over 2 steps) of 12r as a mixture of cis- and trans-olefins.

Step 7. Hydrogenation of 12r to give 13r

Palladium on carbon (10 wt. %, 40 mg) was added to a solution of 12r (163 mg, 0.22 mmol) in EtOAc (2.2 mL). A hydrogen atmosphere was established by evacuating and refilling with hydrogen (5×) and the reaction mixture was stirred under a balloon of hydrogen for 18 h. The reaction mixture was filtered through celite, washing with EtOAc, and the filtrate was concentrated in vacuo to afford 150 mg (92%) of saturated compound 13r.

Step 8. Deprotection of 13r to give 25

2,3-Dichloro-5,6-dicyano-1,4-benzoquinone (DDQ, 43 mg, 0.19 mmol) was added to a mixture of 13r (66 mg, 0.09 mmol) in CHCl₃ (0.95 mL) and water (0.05 mL) at room temperature. After 1 h at room temperature, the reaction was quenched with saturated aqueous NaHCO₃ (10 mL) and extracted with EtOAc (3×15 mL). The combined extracts were washed with saturated aqueous NaHSO₃ (10 mL) and brine (10 mL) then dried (Na₂SO₄), filtered and concentrated in vacuo. The crude residue was purified on 4 g silica (hexanes→60% EtOAc/hexanes, gradient) to afford 44 mg (80%) of alcohol 25.

Step 9. Oxidative deprotection of 25 to give 26

Water (0.03 mL) and ammonium cerium(IV) nitrate (CAN, 20 mg, 0.036 mmol) were added to a solution of 25 (9 mg, 0.015 mmol) in MeCN (0.3 mL). After 1 h at room temperature, the reaction mixture was heated at 50° C. After 2 h at 50° C., the mixture was cooled to room temperature and quenched with saturated aqueous NaHCO₃ (2 mL) and extracted with CH₂Cl₂ (3×5 mL). The combined extracts were washed with saturated aqueous NaHSO₃ (5 mL) then dried (Na₂SO₄), filtered and concentrated in vacuo. The crude residue was purified on 4 g silica (hexanes→EtOAc, gradient) to afford 3.5 mg (52%) of 26.

Step 10. Saponification of 26 to give 27.

Aqueous 1 N lithium hydroxide (0.05 mL, 0.05 mmol) was added to a solution of ester 26 (3.5 mg, 0.0077 mmol) in THF (0.1 mL). After 3 d at room temperature, the volatiles were evaporated under a stream of nitrogen. The residue was diluted with water (1.0 mL), acidified with 1 N aqueous HCl (0.5 mL), and extracted with EtOAc (3×2 mL). The combined extracts were washed with brine (5 mL), dried (Na₂SO₄), filtered and concentrated in vacuo to afford 2.5 mg (74%) of the title compound (27).

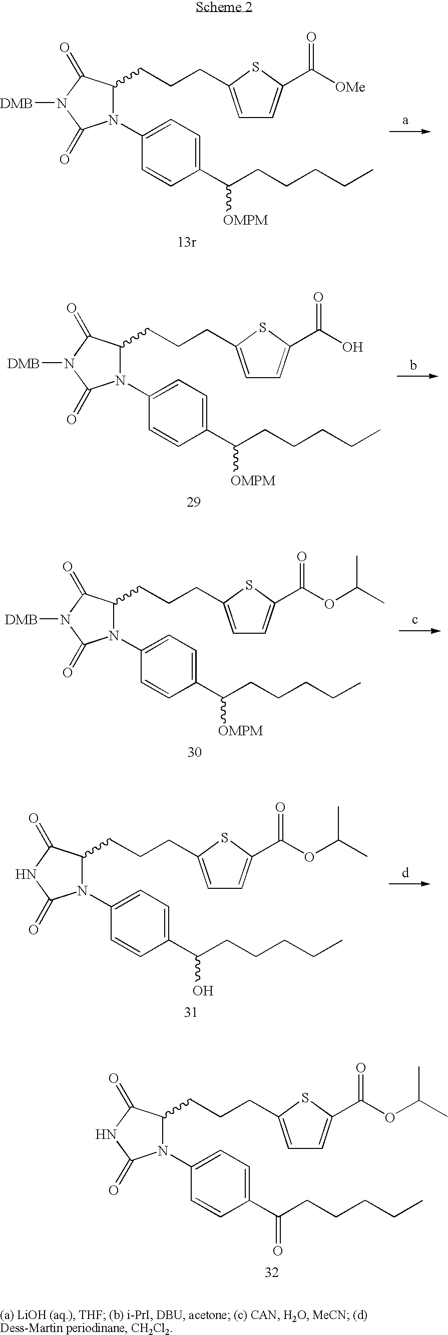

### EXAMPLE 2

### 5-(3-(3-(4-(1-hydroxyhexyl)phenyl)-2,5-dioxoimidazolidin-4-yl)propyl)thiophene-2-carboxylic acid (14r)

Step 1. Reduction of 26 to give 28

Sodium borohydride (1 mg, 0.026 mmol) was added in one portion to a solution of 26 (7 mg, 0.015 mmol) in CH₂Cl₂ (0.1 mL) and MeOH (0.1 mL). After 30 min, the reaction was quenched with saturated aqueous NH₄Cl and extracted with CH₂Cl₂ (3×8 mL). The combined extracts were dried (Na₂SO₄), filtered and concentrated in vacuo. The crude residue was purified on 4 g silica (40% EtOAc/hexanes→EtOAc, gradient) to afford 7 mg (quant.) of 28.

Step 2. Saponification of 28 to give 14r

In accordance with the procedure of Example 1, step 10, ester 28 (7 mg, 0.015 mmol) was converted into 6.5 mg (96%) of the title compound (14r).

### EXAMPLE 3

### Isopropyl 5-(3-(3-(4-(1-hydroxyhexyl)phenyl)-2,5-dioxoimidazolidin-4-yl)propyl)thiophene-2-carboxylate (31)

Step 1. Saponification of 13r to give 29

In accordance with the procedure of Example 1, step 10, ester 13r (90 mg, 0.123 mmol) was converted into 86 mg (97%) of 29 after purification on 4 g silica (CH₂Cl₂→15% MeOH/CH₂Cl₂, gradient).

Step 2. Esterification of 29 to give 30

1,8-Diazabicyclo[5.4.0]undec-7-ene (DBU, 36 μL, 0.24 mmol) and 2-iodopropane (0.40 mL, 4.0 mmol) were added to a solution of acid 29 (86 mg, 0.12 mmol) in acetone (1.2 mL) at room temperature under nitrogen. After 18 h at room temperature, the volatiles were evaporated under a stream of nitrogen. The residue was diluted with water (5 mL), acidified with 1.0 N HCl (5 mL) extracted with EtOAc (3×30 mL). The combined extracts dried (Na₂SO₄), filtered and concentrated in vacuo. Purification of the residue by flash column chromatography on 12 g silica (hexanes→60% EtOAc/hexanes, gradient) afforded 72 mg (79%) of 30.

Step 3. Deprotection of 30 to give 31

Water (0.2 mL) and CAN (209 mg, 0.38 mmol) were added to a solution of 30 (72 mg, 0.095 mmol) in MeCN (1.9 mL). After 4 h at room temperature, the reaction mixture was quenched with saturated aqueous NaHCO₃ (20 mL) and extracted with EtOAc (3×35 mL). The combined extracts were washed with saturated aqueous NaHSO₃ (2×30 mL) and brine (30 mL) then dried (Na₂SO₄), filtered and concentrated in vacuo. The crude residue was purified on 12 g silica (hexanes→EtOAc, gradient) to afford 16 mg (35%) of the title compound (31).

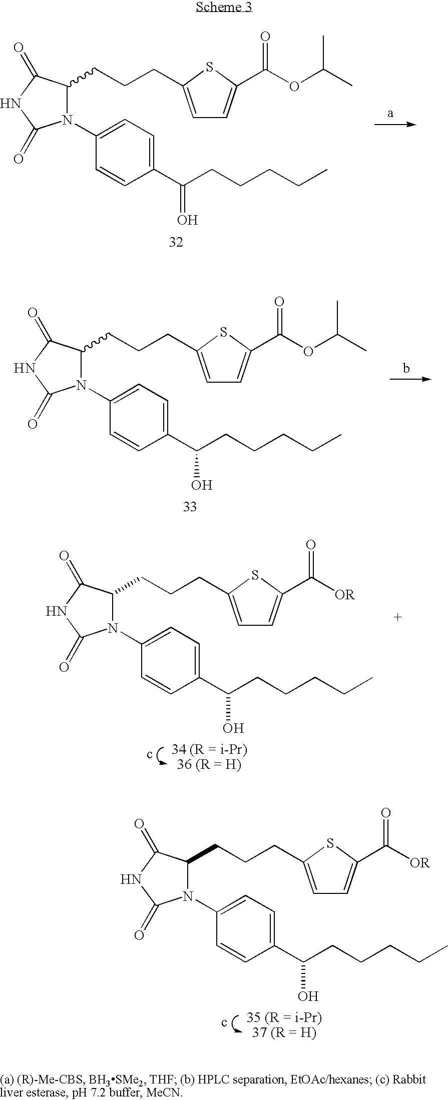

### EXAMPLE 4

### Isopropyl 5-(3-(3-(4-hexanoylphenyl)-2,5-dioxoimidazolidin-4-yl)propyl)thiophene-2-carboxylate (32)

Dess-Martin periodinane (9 mg, 0.021 mmol) was added to a solution of 31 (9 mg, 0.016 mmol) in CH₂Cl₂ (0.16 mL) at room temperature. After 4 h the reaction mixture was diluted with 20% EtOAc/hexanes (2 mL) and filtered through celite, washing with additional 20% EtOAc/hexanes (2 mL). The filtrate was concentrated in vacuo. The crude residue was purified on 4 g silica (hexanes→EtOAc, gradient) to afford 4.5 mg (56%) of the title compound (32).

### EXAMPLES 5 AND 6

### Isopropyl 5-(3-((S)-3-(4-((S)-1-hydroxyhexyl)phenyl)-2,5-dioxoimidazolidin-4-yl)propyl)thiophene-2-carboxylate (34) and isopropyl 5-(3-((R)-3-(4-((S)-1-hydroxyhexyl)phenyl)-2,5-dioxoimidazolidin-4-yl)propyl)thiophene-2-carboxylate (35)

Step 1. Asymmetric reduction of 32 to give 33

BH₃.SMe₂ (0.8 mL of a 5.0 M solution in Et₂O, 4.0 mmol) was added to a solution of (R)-2-methyl-CBS-oxazaborolidine (133 mg, 0.48 mmol) in THF (20 mL). After 15 min at room temperature, a solution of ketone 32 (1.94 g, 4.0 mmol) in THF (20 mL) was added over 30 min. After 15 min further at room temperature, the reaction was quenched over 10 min with MeOH (5 mL). The mixture was diluted with EtOAc (100 mL) and washed with 1.0 M HCl (50 mL) and brine (50 mL), then filtered and concentrated in vacuo. Purification of the residue by flash column chromatography on 50 g silica (15% EtOAc/hexanes→50% EtOAc/hexanes, gradient) afforded 1.65 g (85%) of 33. Chiral HPLC analysis (Chiralpak AD, 3.6×250mm column) showed 33 to have approximately 94% enantiomeric excess.

Step 2. HPLC separation of 33 to give 34 and 35

Mixture of diastereomers 33 was separated on a Waters 600 HPLC instrument employing a Waters 2996 PDA detector and a Phenomenex Luna 10μ prep silica (2) 1 column, 50 mm×250 mm (p/no. 00G-4322-V0; s/no. 356757-1). The flow rate was 45 mL/min, 60% EtOAc/Hex was the eluent and 28 mg of 33 was used in each injection. The first diastereomer (34) eluted at 55-60 min, and the second diastereomer (35) eluted at 60-65 min. Three injections afforded 39 mg of 34 and 41 mg of 35.

### EXAMPLE 7

### 5-(3-((S)-3-(4-((S)-1-hydroxyhexyl)phenyl)-2,5-dioxoimidazolidin-4-yl)propyl)thiophene-2-carboxylic acid (36)

Ester 34 (8 mg, 0.016 mmol) was dissolved in MeCN (0.1 mL) and pH 7.2 buffer (2.0 mL) was added. Rabbit liver esterase (80 units/mg, 6 mg, 480 units) was added and the mixture was stirred vigorously at room temperature. After 5 d, the mixture was diluted with MeCN (25 mL) and concentrated to dryness. Purification of the crude residue by chromatography on 4 g silica gel (CH₂Cl₂→20% MeOH/CH₂Cl₂, gradient) afforded 4.5 mg (62%) of the title compound (36).

### EXAMPLE 8

### 5-(3-((R)-3-(4-((S)-1-hydroxyhexyl)phenyl)-2,5-dioxoimidazolidin-4-yl)propyl)thiophene-2-carboxylic acid (37)

In accordance with the procedure of example 7, ester 35 (8 mg, 0.016 mmol) was converted into 5.0 mg (68%) of the title compound (37).

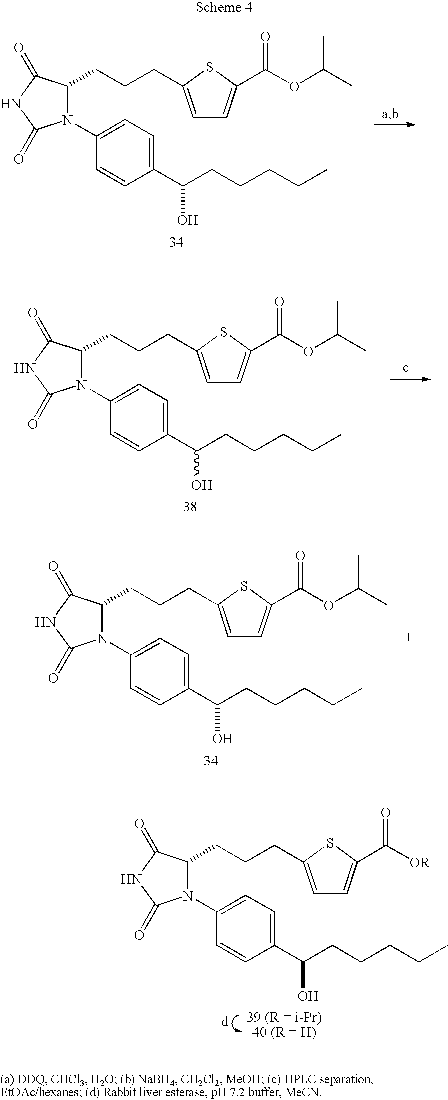

### EXAMPLE 9

### Isopropyl 5-(3-((S)-3-(4-((R)-1-hydroxyhexyl)phenyl)-2,5-dioxoimidazolidin-4-yl)propyl)thiophene-2-carboxylate (39)

Step 1. Oxidation of 34 followed by reduction to give 38

DDQ (22 mg, 0.097 mmol) was added to a mixture of 34 (23 mg, 0.047 mmol) in CHCl₃ (0.48 mL) and water (0.03 mL) at room temperature. After 1 h at room temperature, the reaction was incomplete by tlc analysis. The reaction was heated at 40° C. for 3 h then was cooled and quenched with saturated aqueous NaHCO₃ (10 mL) and extracted with EtOAc (3×10 mL). The combined extracts were washed with saturated aqueous NaHSO₃ (2×10 mL) and brine (10 mL) then dried (Na₂SO₄), filtered and concentrated in vacuo. The crude residue was purified on 12 g silica (hexanes→EtOAc, gradient) to afford 22 mg (96%) of interemediate ketone. A solution of this ketone (22 mg, 0.045 mmol) in CH₂Cl₂ (0.11 mL) and MeOH (0.11 mL) was treated with sodium borohydride (3 mg, 0.079 mmol), which was added in one portion at 0° C. After 1 h, the reaction was quenched with saturated aqueous NH₄Cl (5 mL) and extracted with EtOAc (3×10 mL). The combined extracts were dried (Na₂SO₄), filtered and concentrated in vacuo. The crude residue was purified on 4 g silica (hexanes→EtOAc, gradient) to afford 20 mg (90%) of 38.

Step 2. HPLC separation of 38 to give 34 and 39

Mixture of diastereomers 38 was separated on a Waters 600 HPLC instrument employing a Waters 2996 PDA detector and a Phenomenex Luna 10μ prep silica (2) 1 column, 50 mm×250 mm (p/no. 00G-4322-V0; s/no. 356757-1). The flow rate was 45 mL/min, 50% EtOAc/Hex was the eluent and 20 mg of 38 was used in each injection. The first diastereomer (34, 9 mg) eluted at 64-68 min, and the second diastereomer (39, 8 mg) eluted at 68-74 min.

### EXAMPLE 10

### 5-(3-((S)-3-(4-((R)-1-hydroxyhexyl)phenyl)-2,5-dioxoimidazolidin-4-yl)propyl)thiophene-2-carboxylic acid (40)

In accordance with the procedure of example 7, ester 39 (7 mg, 0.014 mmol) was converted into 5.0 mg (78%) of the title compound (40).

### In Vitro Testing

United States Patent Application Publication No. 20070129552, incorporated by reference herein, describes the methods used to obtain the in vitro data in the table below.

| | | EP2 data | | | EP4 data | | | | | | | |
|---|---|---|---|---|---|---|---|---|---|---|---|---|
| Ex- | | flipr | cAMP | | flipr | | Other Receptors (EC50 in nM) | | | | | |
| ample | Structure | EC50 | EC50 | Ki | EC50 | KI | hFP | hEP1 | hEP3A | hTP | hIP | hDP |
| 1 | 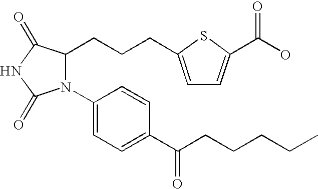 | 17 | 0.2 | 4.5 | 17515 | 3243 | NA | NA | 131 | NA | NA | 8807 |
| 2 | 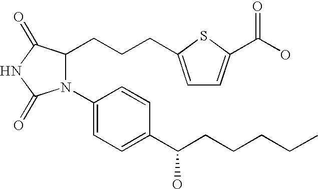 | 3.5 | 0.1 | 5 | NT | >10000 | NA | NA | 10 | NA | NA | 17379 |
| 7 | 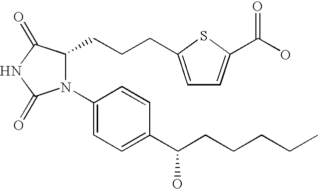 | 4 | 0.02 | 2 | NT | 5067 | NA | NA | 5 | NA | NA | >10000 |
| 8 | 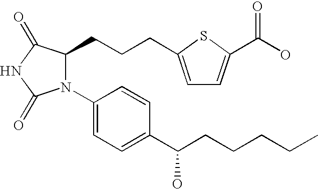 | 23 | 0.2 | 4 | NT | 7185 | NA | NA | 29 | NA | NA | >10000 |
| 10 | 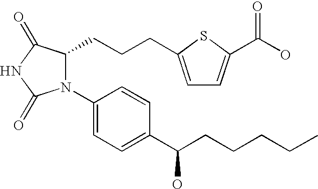 | 1 | 0.03 | 1 | 2000 | 7213 | NA | NA | 15 | NA | NA | 19256 |

### In Vivo Testing

U.S. Pat. No. 7,091,231 describes the methods used to carry out the tests reported below.

Isopropyl 5-(3-(3-(4-(1-hydroxyhexyl)phenyl)-2,5-dioxoimidazolidin-4-yl)propyl)thiophene-2-carboxylate (31) was tested in normotensive dogs at 0.003%, dosing once daily for 5 days. The maximum intraocular pressure (IOP) decrease from baseline was 7.7 mmHg (45%) at 102 h; the maximum ocular surface hyperemia (OSH) score was 2.5 at 26 h. This compound was also tested in laser-induced hypertensive monkeys, using one single day dose. At 0.003%, the maximum IOP decrease from baseline was 23 mmHg (53%) at 24 h.

Isopropyl 5-(3-(3-(4-hexanoylphenyl)-2,5-dioxoimidazolidin-4-yl)propyl)thiophene-2-carboxylate (32) was tested in normotensive dogs at 0.003%, dosing once daily for 5 days. The maximum intraocular pressure (IOP) decrease from baseline was 8.9 mmHg (48%) at 54 h; the maximum ocular surface hyperemia (OSH) score was 2.3 at 54 h. This compound was also tested in laser-induced hypertensive monkeys, using one single day dose. At 0.003%, the maximum IOP decrease from baseline was 19 mmHg (49%) at 24 h.

What is claimed is:

1. A compound represented by a formula:

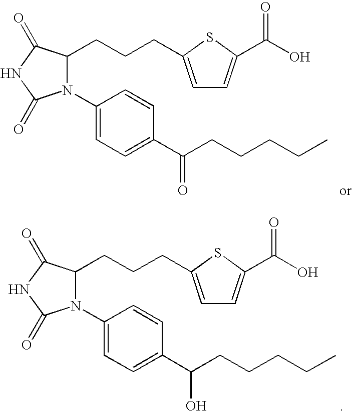

2. The compound of claim 1 represented by a formula:

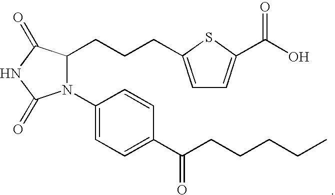

3. The compound of claim 1 represented by a formula:

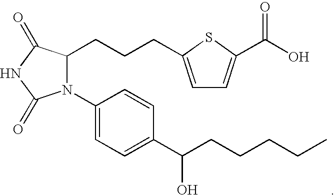

4. The compound of claim 3 represented by a formula:

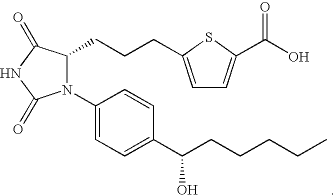

5. The compound of claim 3 represented by a formula:

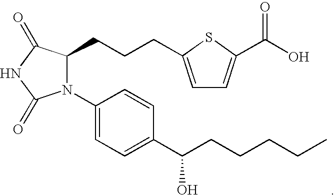

6. The compound of claim 3 represented by a formula:

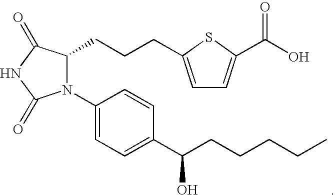

7. A method comprising administering the compound of claim 1 to a mammal for the reduction of intraocular pressure.

8. The method of claim 7 wherein the mammal is a person.

9. The method of claim 7 wherein the compound is represented by a formula:

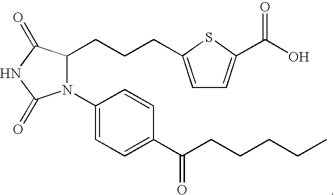

10. The method of claim 7 wherein the compound is represented by a formula:

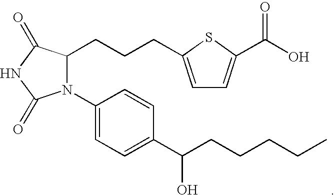

11. The method of claim 7 wherein the compound is represented by a formula:

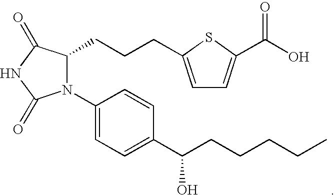

12. The method of claim 7 wherein the compound is represented by a formula:

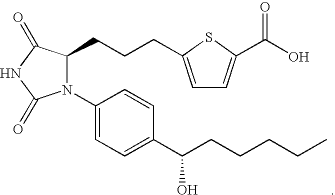

13. The method of claim 7 wherein the compound is represented by a formula:

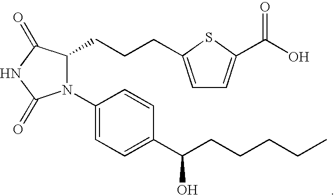

14. A composition comprising a compound according to claim 1 and an ophthalmically acceptable excipient, wherein the composition is a liquid.
